(19) United States
(12) Patent Application Publication
(10) Pub. No.: US 2024/0148672 A1
(43) Pub. Date: May 9, 2024

(54) ORAL THIN FILM

(71) Applicant: LTS LOHMANN THERAPIE-SYSTEME AG, Andernach (DE)

(72) Inventors: Markus MÜLLER, Troisdorf (DE); Mario FICKER, Bonn (DE)

(21) Appl. No.: 18/548,690

(22) PCT Filed: Mar. 2, 2022
(86) PCT No.: PCT/EP2022/055284

§ 371 Date: Sep. 1, 2023

(30) Foreign Application Priority Data
Mar. 4, 2021 (DE) 102021105268.8

(51) Int. Cl. A61K 31/135; A61K 9/00; A61K 47/32

(52) U.S. Cl. CPC A61K 31/135; A61K 9/006; A61K 47/32

(57) ABSTRACT

The present invention relates to an oral thin film comprising at least one matrix polymer and at least one active pharmaceutical ingredient, wherein the at least one active pharmaceutical ingredient is an acid or a base, characterised in that the at least one active pharmaceutical ingredient is present in the form of a mixture which comprises the at least one active pharmaceutical ingredient both in the form of the free acid or base and in the form of a pharmaceutically acceptable salt, a method for producing said oral thin film, and the use of such an oral thin film as a medicament.

The present invention relates to an oral thin film comprising at least one matrix polymer and at least one active pharmaceutical ingredient, wherein the at least one active pharmaceutical ingredient is an acid or a base, and wherein the at least one active pharmaceutical ingredient is present in the form of a mixture which comprises the at least one active pharmaceutical ingredient both in the form of the free acid or base and in the form of a pharmaceutically acceptable salt, a method for producing said oral thin film, and the use of such an oral thin film as a medicament.

Oral thin films are thin films containing at least one active pharmaceutical ingredient that are placed directly in the oral cavity or against the oral mucosa and dissolve or macerate there and in so doing deliver the active ingredient. These films are, especially, thin, one- or multi-layer, active-ingredient-containing polymer-based films which, when applied to a mucous membrane, especially the oral mucosa, can deliver the active ingredient directly into same. The very good blood supply to the oral mucosa ensures a rapid transfer of the active ingredient into the bloodstream. This dosage system has the advantage that the active ingredient is absorbed for the most part by the mucous membrane, thus avoiding the first-pass effect, which occurs in the case of the conventional dosage form of an active ingredient in tablet form. The active ingredient may be dissolved, emulsified or dispersed in the film.

In order for the active pharmaceutical ingredient to be absorbed via a mucous membrane and to ensure the stability of an active pharmaceutical ingredient and the stability of a pharmaceutical composition on the whole, the pH value is a key factor. Especially when dissolving an oral thin film in the mouth of a patient, the resultant pH value at the location where the oral thin film dissolves is decisive for the absorption of the active pharmaceutical ingredient.

Desired pH values are usually set by adding acids or bases or by buffer systems, however, this may have a detrimental effect on the pharmaceutical formulation. By neutralising an active ingredient by adding acid or base to the formulation, a salt is produced as by-product and negatively influences the formulation since it provides no further function. The resultant salt increases especially also the area density of an oral thin film and may additionally negatively influence the stability of the formulation and the taste. The same is true for the use of buffer systems.

The aim of the present invention lies in overcoming the above-mentioned disadvantages of the prior art. Especially, the aim of the present invention lies in providing an oral thin film of which the pH value, especially the pH value once the oral thin film has dissolved, can be set advantageously without having to add further acid, base or buffer systems to the oral thin film. Furthermore, by way of the oral thin film it will be made possible to set the pH value in the patient's saliva within the greatest possible range, moreover in the most stable manner possible. In addition, it will be possible to produce the oral thin film as easily and economically as possible.

The above aim is addressed by a multi-layer oral thin film according to claim 1, especially by an oral thin film comprising at least one matrix polymer and at least one active pharmaceutical ingredient, wherein the at least one active pharmaceutical ingredient is an acid or a base, which oral thin film is characterised in that the at least one active pharmaceutical ingredient is present in the form of a mixture which comprises the at least one active pharmaceutical ingredient both in the form of the free acid or base and in the form of a pharmaceutically acceptable salt.

Such an oral thin film is advantageous since, as a result of the mixture, which comprises the at least one active pharmaceutical ingredient both in the form of the free acid or base and in the form of a pharmaceutically acceptable salt, a buffer system is provided in the oral thin film without having to add further acids, bases or buffer substances. By adjusting the ratio of the at least one active pharmaceutical ingredient in the form of the free acid or base to the at least one active pharmaceutical ingredient in the form of a pharmaceutically acceptable salt, it is possible to adjust the pH value of the formulation. Since no further acid, base or buffer substance has to be added to such a formulation, the formulation and thus the final oral thin film is also not negatively influenced by the creation of salts during the neutralisation or by the presence of a buffer system.

For the definition of acid and base, reference can be made especially to the Bronsted acid-base concept known to a person skilled in the art. The oral thin film according to the invention thus comprises especially at least one active pharmaceutical ingredient which is an acid or a base according to the Bronsted acid-base concept.

In the present document, the word “comprising” can also mean “consisting of”.

The oral thin film according to the invention is further preferably characterised in that the at least one matrix layer comprises a water-soluble polymer.

Water-soluble polymers comprise chemically very different natural or synthetic polymers, the common feature of which is their solubility in water or aqueous media. A precondition is that these polymers have a number of hydrophilic groups sufficient for the water solubility and are not crosslinked. The hydrophilic groups may be non-ionic, anionic, cationic and/or zwitterionic.

Water-soluble is preferably understood to mean a solubility in water of greater than 100 g/L at 25° C.

The at least one water-soluble polymer is preferably selected from the group comprising starch and starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, vinylpyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums, polyvinyl alcohol-polyethylene glycol graft copolymers, with polyvinyl alcohols being especially preferred.

The oral thin film according to the invention is also preferably characterised in that the at least one matrix polymer, preferably the water-soluble polymer, is present in an amount of 10 to 90 wt. %, preferably of 20 to 60 wt. %, especially preferably of 30 to 50 wt. %, in relation to the total weight of the oral thin film.

The oral thin film according to the invention is preferably characterised in that the oral thin film, besides the mixture comprising the at least one active pharmaceutical ingredient both in the form of the free acid or base and in the form of a pharmaceutically acceptable salt, does not comprise any further acids, bases, salts and/or buffer systems.

Especially, the oral thin film does not contain any hydrochloric acid, no NaOH, no KOH, no Na₂CO₃, no NaHCO₃, no K₂CO₃ and/or no sulfuric acid.

Furthermore, the oral thin film according to the invention does not contain any NaCl, KCl, phosphate buffer, TRIS buffer, citrate buffer, carbonate buffer, sulfate buffer, borate buffer and/or ammonium buffer.

The at least one active pharmaceutical ingredient, apart from the fact that it is an acid or a base, is in principle not subject to any restriction, but is preferably selected from all active pharmaceutical ingredients which are suitable for oral and/or transmucosal application.

Preferably, the oral thin film according to the invention is characterised in that the at least one active pharmaceutical ingredient, when it is an acid, has a pKs of 3 to 11, preferably of 4 to 9.

Preferably, the oral thin film according to the invention is characterised in that the at least one active pharmaceutical ingredient, when it is a base, has a pKb of 3 to 11, preferably of 4 to 9.

Preferably the oral thin film according to the invention is characterised in that the at least one active pharmaceutical ingredient comprises a carboxyl group, an amino group, a sulfonyl group and/or a phosphonate group.

Preferred active ingredients are selected from the group comprising the active ingredient classes of analgesics, hormones, hypnotics, sedatives, antiepileptics, analeptics, psychoneurotropic drugs, neuro-muscle blockers, antispasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active ingredients, antibiotics, chemotherapeutics and narcotics, although this group is not exhaustive.

The at least one active pharmaceutical ingredient is especially preferably ketamine.

Ketamine is understood to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyl)-2-(methylamino)cyclohexan-1-one.

(S)-ketamine is especially preferably present as a single stereoisomer of ketamine, since the analgesic and anaesthetic potency of (S)-ketamine is approximately three times higher than that of the (R) form.

Preferably the oral thin film according to the invention is characterised in that the mixture comprising the at least one active pharmaceutical ingredient both in the form of the free acid or base and in the form of a pharmaceutically acceptable salt comprises ketamine as free base and ketamine hydrochloride, preferably (S)-ketamine as free base and (S)-ketamine hydrochloride.

Due to the mixture of ketamine as free base and ketamine hydrochloride, especially suitable pH values can be set without having to add further acids, bases or buffer systems.

The active ingredient content in the oral thin film can vary within relatively wide limits. A range of 10 to 60 wt. %, in relation to the total weight (dry total weight) of the oral thin film, can be stated as suitable. In one embodiment, the proportion of active ingredient in the oral thin film lies rather in the lower range, for example if the active ingredient has a strong unpleasant taste, which has to be compensated for by a greater amount of taste-masking agents. In this case, a range of 10 to 40 wt. % can be stated as suitable active ingredient fraction. In another embodiment, the proportion of active ingredient in the dosage form according to the invention lies rather in the upper range, wherein a content of 40 to 60 wt. % and especially a content of 45 to 55 wt. % can be stated as being especially preferred.

The amount of active pharmaceutical ingredient relates here to the mixture comprising the at least one active pharmaceutical ingredient both in the form of the free acid or base and in the form of a pharmaceutically acceptable salt, that is to say to the sum of the amounts of the at least one active pharmaceutical ingredient in the form of the free acid or base and the amount of the at least one active pharmaceutical ingredient in the form of a pharmaceutically acceptable salt.

Especially preferably the oral thin film according to the invention is therefore characterised in that the mixture comprising the at least one active pharmaceutical ingredient both in the form of the free acid or base and in the form of a pharmaceutically acceptable salt is present in an amount of 35 to 60 wt. %, in relation to the total weight of the oral thin film.

Especially preferably ketamine is present as a pharmaceutically acceptable salt thereof, especially ketamine as free base and as ketamine hydrochloride, preferably (S)-ketamine as free base and as (S)-ketamine hydrochloride, in the sum of an amount of 35 to 60 wt. %, in relation to the total weight of the oral thin film.

For setting a pH value, the molar ratio of the at least one active pharmaceutical ingredient in the form of the free acid or base to the at least one active pharmaceutical ingredient in the form of a pharmaceutically acceptable salt is decisive.

The oral thin film according to the invention is therefore preferably characterised in that the mixture comprising the at least one active pharmaceutical ingredient both in the form of the free acid or base and in the form of a pharmaceutically acceptable salt comprises the at least one active pharmaceutical ingredient in the form of the free acid or base and the at least active pharmaceutical ingredient in the form of a pharmaceutically acceptable salt in a molar ratio of 3:1 to 1:3, preferably 1.5:1 to 1:1.5, especially preferably approximately 1:1, and very especially preferably 1:1.

The molar ratio of ketamine as free base and ketamine as pharmaceutically acceptable salt is preferably 3:1 to 1:3, preferably 1.5:1 to 1:1.5, especially preferably approximately 1:1, and very especially preferably 1:1.

Very especially preferably the oral thin film according to the invention comprises ketamine as free base and ketamine hydrochloride in a molar ratio of 3:1 to 1:3, preferably 1.5:1 to 1:1.5, especially preferably approximately 1:1, and very especially preferably 1:1.

In another preferred embodiment, the oral thin film according to the invention comprises (S)-ketamine as free base and (S)-ketamine hydrochloride in a molar ratio of 3:1 to 1:3, preferably 1.5:1 to 1:1.5, especially preferably approximately 1:1, and very especially preferably 1:1.

The oral thin film according to the invention is preferably further characterised in that the oral thin film has a pH of 3.5 to 9.5, preferably of 4.5 to 8.8.

This is understood to mean that the pH value (at 20° C.) that establishes in a solution prepared by dissolving the oral thin film in pure water.

The multi-layer oral thin film according to the invention is further preferably characterised in that the matrix layer comprises at least one auxiliary substance selected from the group comprising colouring agents, flavourings, sweeteners, plasticisers, taste-masking agents, emulsifiers, enhancers, pH regulators, humectants, preservatives and/or antioxidants.

Each of these auxiliary substances is preferably contained in this layer in an amount of 0.1 to 40 wt. %, preferably of 0.1 to 30 wt. %, especially preferably of 0.1 to 15 wt. %, very especially preferably of 0.1 to 10 wt. %, or 0.1 to 5 wt. %, in relation to the total weight of the matrix layer.

The multi-layer oral thin film according to the invention is, in principle, not limited in the number of layers contained.

For example, embodiments are also conceivable in which the oral thin film according to the invention comprises several active-ingredient-containing layers.

In one embodiment the oral thin film according to the invention may be a substantially smooth film.

The oral thin film according to the invention is preferably characterised in that it is present in the form of a solidified foam that has voids.

The voids and the associated larger surface area of the films facilitate especially the access of water or saliva or other bodily fluids into the interior of the dosage form and thus accelerate the dissolution of the dosage form and the release of the active ingredient.

In the case of a rapidly absorbing active ingredient, transmucosal absorption can also be improved by the rapid dissolution of the matrix layer.

On the other hand, the wall thickness of said voids is preferably small, as these represent solidified bubbles, for example, so that rapid dissolution or destruction of these voids takes place.

A further advantage of this embodiment is that, despite the comparatively high area density, faster drying can be achieved by formulating as a foam than with a comparable non-foamed composition.

The oral thin film according to the invention is preferably characterised in that the voids are isolated from one another and are preferably present in the form of bubbles, the voids being filled with air or a gas, preferably with an inert gas, especially preferably with nitrogen, carbon dioxide, helium or a mixture of at least two of these gases.

According to another embodiment, it is provided that the voids are connected to one another, preferably by forming a continuous channel system penetrating the matrix.

Said voids preferably have a volume fraction of 5 to 98%, preferably of 40 to 80%, in relation to the total volume of the matrix layer. In this way, the advantageous effect of accelerating the dissolution of the thin film is influenced favourably.

Furthermore, surface-active substances or surfactants can be added to the oral thin film for foam formation or to the obtained foam before or after the drying in order to improve the stability of the foam before or after the drying.

Another parameter that influences the properties of the oral thin film according to the invention is the diameter of the voids or bubbles. The bubbles or voids are preferably created with the aid of a foam whipping machine, with which the diameter of the bubbles can be adjusted in a wide range, almost arbitrarily. The diameter of the bubbles or voids can thus lie in the range of 0.01 to 60 μm. The diameter especially preferably lies in the range of 10 and 50 μm.

The oral thin film according to the invention preferably has an area of 0.5 cm² to 10 cm², especially preferably 1.5 cm² to 8 cm².

The area density of the oral thin film according to the invention is preferably at least 10 g/m², more preferably at least 20 g/m², or at least 30 g/m², or most preferably at least 50 g/m², or less than or equal to 400 g/m², more preferably less than or equal to 350 g/m², or less than or equal to 300 g/m², or most preferably less than or equal to 150 g/m². Preferably, the area density is 10 to 400 g/m², more preferably 20 to 350 g/m², or 30 to 300 g/m², most preferably 50 to 200 g/m².

Preferably, the oral thin film according to the invention has a thickness of approximately 10 μm to approximately 500 μm, especially preferably of approximately 20 μm to approximately 300 μm.

The present invention also relates to a method for producing the oral thin film according to the invention, the method comprising the steps of:
a) producing a solution, dispersion or melt containing at least the at least one matrix polymer and the at least one active pharmaceutical ingredient in the form of a mixture comprising the at least one active pharmaceutical ingredient both in the form of the free acid or base and in the form of a pharmaceutically acceptable salt,
b) spreading the solution, dispersion or melt from step a) in order to obtain a film, and
c) drying the film from step b) in order to obtain an oral thin film.

The method according to the invention also preferably fails to comprise a step in which the at least one active pharmaceutical ingredient is fully or partially neutralised by adding acid or base.

The method according to the invention is preferably also characterised in that there is no further buffer system present in the oral thin film.

The method according to the invention preferably comprises an optional step al) comprising the foaming of the solution, dispersion or melt from step a) by introducing a gas or gas mixture, by chemical gas generation or by expansion of a dissolved gas. It is clear to a person skilled in the art that step al) is necessary only if the oral thin film is to be provided in the form of a solidified foam that has voids.

In addition, the present invention relates to an oral thin film, as described above or obtainable by the above-described method, as a medicament.

The present invention additionally relates to an oral thin film, as described above or obtainable by the above-described method, wherein a mixture of ketamine as free base and ketamine as pharmaceutically acceptable salt, preferably ketamine as free base and ketamine hydrochloride, especially (S)-ketamine as free base and (S)-ketamine as pharmaceutically acceptable salt, especially preferably (S)-ketamine as free base and (S)-ketamine hydrochloride, is used, for use in the treatment of pain and/or depression, especially to reduce the risk of suicide and/or for use as a general anaesthetic, preferably to initiate and carry out general anaesthesia, or as a supplement in the case of local anaesthesia and/or as an analgesic.

The preferred embodiments described above for the multi-layer oral thin film according to the invention are also applicable for the method according to the invention, the multi-layer oral thin film obtained by this method, and use thereof as a medicament.

The invention will be explained in greater detail hereinafter on the basis of non-limiting examples.

### EXAMPLES

In the development of oral thin films containing ketamine as active pharmaceutical ingredient, it was found that the pH value in the patient's oral cavity can have an influence on the release of the active ingredient. In order to bring the ketamine hydrochloride used, which in the tested formulation (see Table 1) has a pH of 5.32, into a partly neutralised state, it was mixed with NaOH. This led to the formation of NaCl, which impairs the formulation since it has to be taken into consideration in the composition. When formulating a mixture of ketamine as free base and ketamine hydrochloride in a molar ratio of 1:1, it was possible to achieve substantially the same pH value as with an equimolar 50% neutralisation with NaOH.

TABLE 1

| | | Formulation [wt. %] | | | |
|---|---|---|---|---|---|
| Ingredient | Function | 1 | 2 | 3 | 4 |
| (S)-ketamine | Active ingredient | 50.00 | 50.00 | — | 25.00 |
| HCl | | | | | |
| (S)-ketamine | Active ingredient | — | — | 43.35 | 21.68 |
| base | | | | | |
| Polyvinyl | Matrix polymer | 39.10 | 35.85 | 46.15 | 42.82 |
| alcohol | | | | | |
| NaOH | pH regulator | — | 3.65 | — | — |
| Saccharin Na | Taste corrector | 1.00 | 1.00 | 1.00 | 1.00 |
| Sucralose | Taste corrector | 2.00 | 2.00 | 2.00 | 2.00 |
| Cherry Flavour | Taste corrector | 3.00 | 3.00 | 3.00 | 3.00 |
| Glycerol | Humectant | 4.50 | 2.71 | 4.50 | 4.50 |
| FD&C red | Colouring agent | 0.40 | — | — | — |
| pH- | Measured as | 5.32 | 6.15 | 7.97 | 6.11 |
| value | dissolved OTF in | | | | |
| | demineralised water | | | | |
| | temperature- | | | | |
| | controlled to 32° C. | | | | |
| | (blank value pH | | | | |
| | 7.12). | | | | |
| pH- | Measured as | 5.49 | 6.43 | 7.32 | 6.50 |
| value | dissolved OTF in | | | | |
| | human saliva | | | | |
| | temperature- | | | | |
| | controlled to 32° C. | | | | |
| | (blank value pH | | | | |
| | 6.95) | | | | |

Oral thin films of the composition according to Table 1 were prepared as follows. The active ingredient or the active ingredient mixture was presented and an aqueous solution of the matrix polymer was added. The remaining ingredients were then stirred in. The solution was spread and dried in order to obtain an oral thin film.

It should be noted that the mixture of (S)-ketamine (free base) and (S)-ketamine hydrochloride (4) has substantially the same pH value as the neutralised formulation (2).

Advantages of formulation (4), comprising (S)-ketamine (free base) and (S)-ketamine hydrochloride without addition of NaOH are:

No salt formation by the neutralisation and no associated negative effects.

Due to the low molecular weight of (S)-ketamine (free base), the proportion of active ingredient used is smaller, i.e. proportions of other components can be increased relative thereto.

(S)-ketamine (free base) and the corresponding protonated compound form a buffer system.

(S)-ketamine (free base) is additionally stabilised by the HCl.

Potential improvement in taste, since the pH value may have an influence on the taste (acidic or basic (=soapy)).

By varying the amount of (S)-ketamine (free base) and (S)-ketamine hydrochloride, any pH value between that of the base and the salt can be set.

1. An oral thin comprising at least one matrix polymer and at least one active pharmaceutical ingredient, wherein the at least one active pharmaceutical ingredient is an acid or a base, characterised in that the at least one active pharmaceutical ingredient is present in the form of a mixture which comprises the at least one active pharmaceutical ingredient both in the form of the free acid or base and in the form of a pharmaceutically acceptable salt, wherein the mixture comprising the at least one active pharmaceutical ingredient both in the form of the free acid or base and in the form of a pharmaceutically acceptable salt comprises the at least one active pharmaceutical ingredient in the form of the free acid or base and the at least one active pharmaceutical ingredient in the form of a pharmaceutically acceptable salt of the free acid or base in a molar ratio of 3:1 to 1:3.

2. The oral thin film according to claim 1, characterised in that the at least one matrix polymer comprises a water-soluble polymer.

3. The oral thin film according to claim 1, characterised in that the at least one matrix polymer is selected from the group comprising starch and starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, polyvinyl alcohol-polyethylene glycol graft copolymers, vinylpyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums.

4. The oral thin film according to claim 1, characterised in that the at least one matrix polymer is present in an amount of 10 to 90 wt. %, in relation to the total weight of the oral thin film.

5. The oral thin film according to claim 1, characterised in that the oral thin film, besides the mixture comprising the at least one active pharmaceutical ingredient both in the form of the free acid or base and in the form of a pharmaceutically acceptable salt, does not comprise any further acids, bases, salts and/or buffer systems.

6. The oral thin film according to claim 1, characterised in that the at least one active pharmaceutical ingredient comprises a carboxyl group, an amino group, a sulfonyl group and/or a phosphonate group.

7. The oral thin film according to claim 1, characterised in that the at least one active pharmaceutical ingredient is selected from the group comprising the active ingredient classes of analgesics, hormones, hypnotics, sedatives, antiepileptics, analeptics, psychoneurotropic drugs, neuro-muscle blockers, antispasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active ingredients, antibiotics, chemotherapeutics and narcotics.

8. The oral thin film according to claim 1, characterised in that the at least one active pharmaceutical ingredient comprises ketamine, especially preferably (S)-ketamine.

9. The oral thin film according to claim 1, characterised in that the mixture comprising the at least one active pharmaceutical ingredient both in the form of the free acid or base and in the form of a pharmaceutically acceptable salt comprises ketamine as free base and ketamine hydrochloride, preferably (S)-ketamine as free base and (S)-ketamine hydrochloride.

10. The oral thin film according to claim 1, characterised in that the mixture comprising the at least one active pharmaceutical ingredient both in the form of the free acid or base and in the form of a pharmaceutically acceptable salt is present in an amount of 35 to 55 wt. %, in relation to the total weight of the oral thin film.

11. The oral thin film according to claim 1, characterised in that the mixture comprising the at least one active pharmaceutical ingredient both in the form of the free acid or base and in the form of a pharmaceutically acceptable salt comprises the at least one active pharmaceutical ingredient in the form of the free acid or base and the at least active pharmaceutical ingredient in the form of a pharmaceutically acceptable salt in a molar ratio of 3:1 to 1:31.5:1 to 1:1.5.

12. The oral thin film according to claim 1, characterised in that the oral thin film has a pH of 3.5 to 9.5.

13. The oral thin film according to claim 1, characterised in that the oral thin film further comprises at least one auxiliary selected from the group comprising colouring agents, flavourings, sweeteners, plasticisers, taste-masking agents, emulsifiers, enhancers, humectants, preservatives and/or antioxidants.

14. A method for producing an oral thin film according to claim 1, comprising the steps of:
a) producing a solution, dispersion or melt containing at least the at least one matrix polymer and the at least one active pharmaceutical ingredient in the form of a mixture comprising the at least one active pharmaceutical ingredient both in the form of the free acid or base and in the form of a pharmaceutically acceptable salt, wherein the mixture comprising the at least one active pharmaceutical ingredient both in the form of the free acid or base and in the form of a pharmaceutically acceptable salt contains the at least one active pharmaceutical ingredient in the form of the free acid or base and the at least one active pharmaceutical ingredient in the form of a pharmaceutically acceptable salt of the free acid or base in a molar ratio of 3:1 to 1:3,
b) spreading the solution, dispersion or melt from step a) in order to obtain a film, and
c) drying the film from step b) in order to obtain an oral thin film.

15. A method of administering a medicament comprising providing the oral thin film according to claim 1 to an oral cavity or against an oral mucosa.

16. The oral thin film according to claim 1, characterised in that the oral thin film has a pH of 4.5 to 8.8.
